(19) United States
(12) Patent Application Publication
(10) Pub. No.: US 2023/0366043 A1
(43) Pub. Date: Nov. 16, 2023

(54) METHOD FOR DETECTION OF RNA OR DNA FROM BOLOGICAL SAMPLES

(71) Applicant: MATERIALS AND MACHINES CORPORATION OF AMERICA, LINCOLN, NE (US)

(72) Inventors: ABRAHAM OOMMEN, LINCOLN, NE (US); HEATHER PISCATELLI, LINCOLN, NE (US); SETH LEWIN, LINCOLN, NE (US); GOVARDHAN RATHNAIAH, LINCOLN, NE (US); ALYSSA HANGMAN, LINCOLN, NE (US)

(73) Assignee: MATERIALS AND MACHINES CORPORATION OF AMERICA, LINCOLN, NE (US)

(21) Appl. No.: 18/030,480

(22) PCT Filed: Oct. 6, 2021
(86) PCT No.: PCT/US21/53849

§ 371 Date: Apr. 5, 2023

Related U.S. Application Data
(60) Provisional application No. 63/088,347, filed on Oct. 6, 2020.

(51) Int. Cl. C12Q 1/70; C12Q 1/6883; C12Q 1/6844

(52) U.S. Cl. CPC C12Q 1/701; C12Q 1/6883; C12Q 1/6844; C12Q 1/6806

(57) ABSTRACT

A method of detection of nucleic acids from a biological sample without isolation or purification of the nucleic acids is described. The method may include direct detection of nucleic acids from humans, animals, viruses or bacteria, including DNA and RNA from a biological sample without isolating or purifying nucleic acids prior to analysis. Biological samples may be blood, urine, semen, tissue, swabs (nasal, buccal, ocular, vaginal or anal).

This application claims the benefit of priority to U.S. Provisional Patent Application No. 63/088,347 filed Oct. 6, 2020, entitled “METHOD FOR DETECTION OF RENA OR DNA FROM BIOLOGICAL SAMPLES” which is incorporated by reference in its entirety.

### BACKGROUND

The rapid detection of pathogen nucleic acids (i.e. DNA or RNA) in biological samples are a significant need. Presently, due to increase in pandemic situations (e.g. Covid-19 pandemic) rapid identification of pathogens including virus, bacteria and fungi are in high demand. Many conventional methods available for the detection of nucleic acids require purification or isolation (i.e. separation of the nucleic acids from other cellular components for analysis through centrifuge or magnetic beads) of the nucleic acids prior to detection or identification of nucleic acids in an assay. For example, conventional PCR (polymerase chain reaction) techniques include the use of centrifuges or magnetic beads to isolate nucleic acids from other cellular components and debris prior to amplification and detection. Therefore, it is desirable for a method of nucleic acid detection that does not require isolation or purification of the nucleic acids prior to detection or identification of the nucleic acid.

### SUMMARY

In aspects of the invention the method of direct human, animal, microbial, and viral nucleic acid detection from a biological sample without isolation or purification of the nucleic acids prior to detection of the nucleic acids, the method includes contacting a collected biological sample with a treatment buffer; heating the collected biological sample contacted with the treatment buffer from 80 to 95 degrees Celsius from 2 to 10 minutes; analyzing the biological sample for the nucleic acid. In this aspect, the collected biological sample may be a nasal swab from a human collected into a volume of viral transport media solution, where the nucleic acid analyzed detects the Sars-Cov2 ribonucleic acid virus; the treatment buffer includes 10% Bovine Serum Albumin, 0.4 mM ethylene diamine tetra acetic acid of pH 8, 48 mM Guanidine isothiocyanate, and 8 mM Tris(hydroxymethyl)aminomethane hydrochloride of pH 9.0 of a volume equal to the volume of collected biological sample; and the heating includes heating the collected biological sample contacted with the treatment buffer at 95 degrees Celsius for 2 minutes. In this aspect, the collected biological sample may be an oral swab from a human collected into 500 microliters of the treatment buffer, where the nucleic acid analyzed detects the Sars-Cov2 ribonucleic acid virus; the treatment buffer includes 0.25 mM Sodium Citrate pH 6.7 and 2 mM Tris(2-carboxyethyl)phosphine hydrochloride, and the heating includes heating the collected biological sample contacted with the treatment buffer at 95 degrees Celsius for 2 minutes. In this aspect, the collected biological sample may be an oral fluid sample of a porcine chew rope, where the nucleic acid analyzed detects the porcine reproductive and respiratory syndrome ribonucleic acid virus, wherein the collected biological sample is centrifuged at 2,000× gravity in a mini centrifuge for 10 minutes; the treatment buffer includes a volume equal to a volume of the collected biological sample 2 mM 1,2-Cyclohexanedinitrilotetraacetic acid of pH 8, from 4 mM diethylenetriaminepentaacetic acid of pH 8, 1 mM ethylene diamine tetra acetic acid of pH 8, 5 mM Sodium Citrate of pH 6.5, 2 mM Tris(2-carboxyethyl)phosphine hydrochloride, and the heating includes heating the collected biological sample contacted with the treatment buffer at 80 degrees Celsius for 10 minutes. In this aspect, the collected biological sample may be 50 microliters of porcine serum where the nucleic acid analyzed detects the porcine reproductive and respiratory syndrome ribonucleic acid virus; the treatment buffer includes 50 microliters of 2 mM 1,2-Cyclohexanedinitrilotetraacetic acid of pH 8, 4 mM diethylenetriaminepentaacetic acid of pH 8, 1 mM ethylene diamine tetra acetic acid of pH 8, 1 mM ethylene glycol-bis(3-aminoethyl ether)-N,N,N′,N′-tetraacetic acid of pH 8, 10 mM Tris(hydroxymethyl)aminomethane hydrochloride of pH 9.0, and the heating includes heating the collected biological sample contacted with the treatment buffer at 95 degrees Celsius for 2 minutes. In this aspect, the collected biological sample may be 50 microliters of porcine processing fluid where the nucleic acid analyzed detects the porcine reproductive and respiratory syndrome ribonucleic acid virus; the treatment buffer includes 50 microliters of 3 mM magnesium chloride, 75 mM potassium chloride, 50 mM Tris(hydroxymethyl)aminomethane hydrochloride of pH 9.0, and from 5.0 mM mM Tris(2-carboxyethyl)phosphine hydrochloride, where the treatment buffer is adjusted to pH 8.3; and the heating includes heating the collected biological sample contacted with the treatment buffer at 95 degrees Celsius for 2 minutes. In this aspect, the collected biological sample may be an oral swab from a human collected into a volume of universal transport media, where the nucleic acid analyzed is a hemochromatosis (HFE) gene having a G63D mutation; the treatment buffer includes a volume equal to the volume of universal transport media comprising 0.5 mM ethylene diamine tetra acetic acid of pH 8, 80 mM Guanidine isothiocyanate, and 10 mM Tris(hydroxymethyl)aminomethane hydrochloride of pH 9.0; and the heating includes heating the collect biological sample contacted with the treatment buffer at 95 degrees Celsius for 2 minutes.

In aspects of the invention a method of direct human, animal, microbial, and viral nucleic acid detection from a biological sample without isolation or purification of nucleic acids prior to detection of the nucleic acids, the method includes contacting a collected biological sample with a treatment buffer, the treatment buffer comprising at least one chelating agent and at least one buffering agent; heating the collected biological sample contacted with the treatment buffer from 80 to 95 degrees Celsius from 2 to 10 minutes; analyzing the biological sample for the nucleic acid. In the aspect the chelating agent is selected from the group consisting of from 0.4 to 1 milliMolar (mM) ethylene diamine tetra acetic acid of pH 8.0, 2 mM 1,2-Cyclohexanedinitrilotetraacetic acid of pH 8, from 2-4 mM diethylenetriaminepentaacetic acid of pH 8, from 0.25 to 5 mM Tris(2-carboxyethyl)phosphine hydrochloride, and 1 mM ethylene glycol-bis(β-aminoethyl ether)-N,N,N′,N′-tetraacetic acid, and combinations thereof. In this aspect, the buffering agent is selected from the group consisting of from 8 to 50 mM Tris(hydroxymethyl)aminomethane hydrochloride, 3 mM magnesium chloride, and 75 mM potassium chloride, 5 mM sodium citrate of pH from 6.5 to 6.7, and combinations thereof. In this aspect, the treatment buffer may further include a lysis agent selected from the group consisting of from 48 to 250 mM guanidine isothicynate, 2 mM Tris(2-carboxyethyl)phosphine hydrochloride, and combinations thereof.

### FIGURES

FIG. 1 represents a treatment buffer for use in a method for direct nucleic acid detection from a biological sample without isolation or purification of nucleic acids prior to detection or identification of the nucleic acids.

FIG. 2 represents a method for nucleic acid detection from a portion of a biological sample using the treatment buffer and without isolation or purification of nucleic acids prior to detection or identification of the nucleic acids.

FIG. 3 is an example that demonstrates the efficacy of the method 200 deploying a treatment buffer for detecting the presence of the Sars-Cov2 RNA virus from a human naso-pharyngeal sample as a biological sample.

FIG. 4 is an example that demonstrates the efficacy of the method 200 using a treatment buffer for detecting the presence of the Sars-Cov2 RNA virus from a human naso-pharyngeal sample as a biological sample.

FIG. 5 is an example that demonstrates the efficacy of the method 200 deploying a treatment buffer for detecting the presence of the porcine reproductive and respiratory syndrome, also called betaarterivirus suid 1 (referred to as PRRS) RNA virus from an oral fluid sample from a pig as a biological sample.

FIG. 6 is an example that demonstrates the efficacy of the method 200 deploying a treatment buffer for detecting the presence of the PRRS RNA virus from a serum sample from a pig as a biological sample.

FIG. 7 is an example that demonstrates the efficacy of the method 200 deploying a treatment buffer for detecting the presence of the PRRS RNA virus from a processing fluid sample from a pig as a biological sample.

FIG. 8 is an example demonstrates the efficacy of the method 200 deploying a treatment buffer for detecting the presence of hemochromatosis (HFE) and sickle cell anemia (HBB) genes of human DNA using oral swabs as a biological sample.

### DETAILED DESCRIPTION

A method of detection of nucleic acids from a biological sample without isolation or purification of the nucleic acids is described. The method may include direct detection of nucleic acids from a biological sample without isolating or purifying nucleic acids (i.e. without isolation or purification of nucleic acids from other cellular components through centrifuge or magnetic beads, or without two or more centrifuge steps, with one centrifuge step separating cells containing nucleic acids for analysis from a supernatant and a second centrifuge step separating cellular debris from nucleic acids) prior to analysis. The method may include detection of nucleic acids from a biological sample using a treatment buffer and without further isolation or purification of the nucleic acids. Biological samples may be blood, urine, tissue, swabs (nasal, buccal, ocular, vaginal or anal).

The following terms have the their assigned meaning as used in the application:
EDTA means ethylene diamine tetra acetic acid
EGTA means ethylene glycol-bis(β-aminoethyl ether)-N,N,N′,N′-tetraacetic acid
DCTA means 1,2-Cyclohexanedinitrilotetraacetic acid
DTPA means diethylenetriaminepentaacetic acid
TCEP-HCl means Tris(2-carboxyethyl)phosphine hydrochloride
Tris HCl means Tris(hydroxymethyl)aminomethane hydrochloride

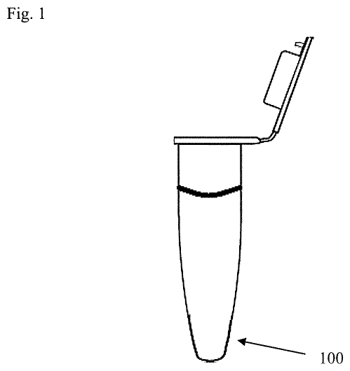

FIG. 1 represents a treatment buffer 100 for use in a method for nucleic acid detection from a biological sample using the treatment buffer without isolation or purification of nucleic acids prior to detection or identification of the nucleic acid. The treatment buffer includes at least one chelating agent and at least one buffer agent. The treatment buffer may further include a lysis agent.

The at least one chelating agent of the treatment buffer stabilizes the released nucleic acids of the biological sample and interacts with other cellular components and cellular debris contained in the sample to facilitate analyzing the nucleic acids without isolation or purification of the nucleic acids. The chelating agent may be selected from the group consisting of from 0.4 to 1 milliMolar (mM) EDTA of pH 8.0, 2 mM DCTA of pH 8, from 2-4 mM DTPA of pH 8, from 0.25 to 5 mM TCEP-HCl, and 1 mM EGTA, and combinations thereof.

The at least one buffering agent of the treatment buffer stabilizes the biological sample by maintaining a pH of the treatment buffer contacted biological sample from between pH 5 to 9. The buffering agent may be selected from the group consisting of from 8 to 50 mM Tris HCl, 5 mM sodium citrate of pH from 6.5 to 6.7, 3 mM magnesium chloride, and 75 mM potassium chloride, and combinations thereof.

The treatment buffer may further include a lysis agent to further facilitate lysis of the cellular membrane, nucleus, and protein coat in the case of a virus to further facilitate release of the nucleic acids of the biological sample into the treatment buffer. The lysis agent includes from 48 to 250 mM guanidine isothicynate and 2 mM TCEP-HCl and combinations thereof.

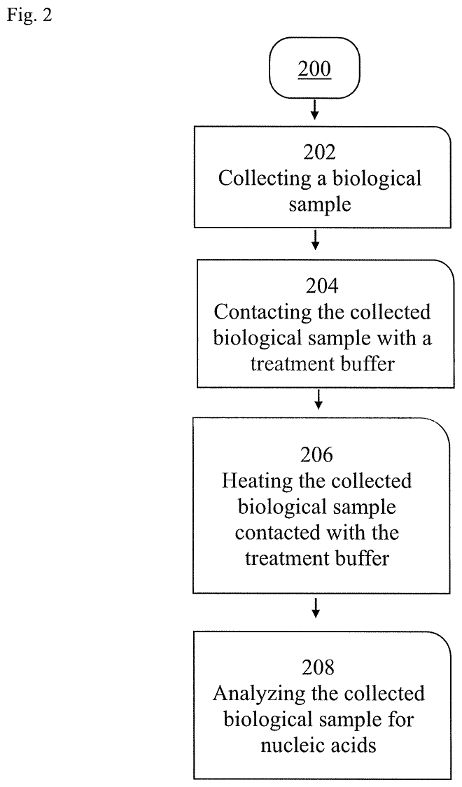

FIG. 2 represents a method for nucleic acid detection from a biological sample using a treatment buffer and without isolation or purification of nucleic acid prior to detection or identification of the nucleic acid. In 202, a biological sample is collected. For example, the biological sample may be a nasal or oral swab, an oral sample, such as a chew rope from pigs, a serum sample from pigs, or a processing fluid sample (i.e. serosanguinous fluid recovered from piglet castrations and tail dockings). The collection may include collection directly into a universal transport media (UTM) or viral transport media (VTM) to stabilize and prevent degradation of the nucleic acids of the biological sample, such as in the case of a viral RNA. The collection of the biological sample may be through conventional mechanisms associated with the biological sample types. The collecting may further include centrifuging the biological sample to separate non-cellular debris (e.g. pieces of chew rope, food, and the like) from the cellular components of the biological sample. The collecting may further include storing the biological sample, such as through freezing and other conventional mechanisms prior to treatment with the treatment buffer.

In 204, the collected biological sample is treated with a treatment buffer. When the biological sample is collected into a VTM or UTM, the treatment buffer is used in a volume equal to the collected biological sample volume. When the biological sample is collected directly into the treatment buffer a volume of 400-600 microliters may be used.

The treatment buffer is selected based on the biological sample. For example, when the biological sample is a nasal swab the treatment buffer may include 0.25 mM Sodium Citrate pH 6.7 and 2 mM TCEP-HCl.

For example, when the biological sample is a nasal or oral swab the treatment buffer may include from 0 to 10% Bovine Serum Albumin, from 0.4 mM to 0.5 mM EDTA of pH 8, from 48 mM to 80 mM Guanidine isothiocyanate, and from 8 to 10 mM Tris-HCl of pH 9.0.

When the biological sample is an oral fluid sample, such as the chew rope from pigs, the treatment buffer may include 2 mM DCTA of pH 8, from 2 to 4 mM DTPA of pH 8, 1 mM EDTA of pH 8, 5 mM Sodium Citrate of pH 6.5, 2 mM TCEP-HCl.

When the biological sample is a serum sample the treatment buffer may include 2 mM DCTA of pH 8, from 2 to 4 mM DTPA of pH 8, 1 mM EDTA of pH 8, 1 mM EGTA of pH 8, 10 mM Tris HCl of pH 9.0.

When the biological sample is a processing fluid from pigs, the treatment buffer may include 3 mM magnesium chloride, 75 mM potassium chloride, 50 mM Tris HCl of pH 9.0, and from 0.25 mM to 5.0 mM TCEP, where the buffer is adjusted to pH 8.3, and combinations thereof.

In 206, the collected biological sample contacted with the treatment buffer is heated. The heating may include heating the collected biological sample contacted with the treatment buffer at from 80 to 95 degrees Celsius for from 2 to 10 minutes. The heating causes cell or protein capsule lysis to further release the nucleic acids into the treatment buffer.

In 208, the biological sample contacted with the treatment buffer and having been heated is analyzed for microbial, viral, human, or animal nucleic acid detection using conventional nucleic acid detection and identification methods, such as polymerase chain reaction (PCR), reverse transcriptase (RT) PCR, real-time PCR, quantitative PCR, isothermal amplification, such as combined sequence amplification and nucleotide detection using the padlock probe as described in U.S. patent application Ser. No. 16/642,308 titled REACTION CONDITIONS COMPOSIION FOR CIRCULARIZING OGLIGONUCELEOTIDE PROBES (referred to herein as a C-SAND® analysis), or the like. For example, when the analysis is real-time PCR with florescence detection or C-SAND analysis with fluorescence detection the device to carry out the analysis may be the device as described in U.S. Pat. Nos. 9,568,429 and 9,810,631 titled WAVELENGTH SCANNING APPARATUS AND METHOD OF USE THEREOF.

The analysis step may include a centrifuge step prior to nucleic acid detection that simultaneously separates non-cellular particulate matter and cellular debris from the nucleic acids for detection.

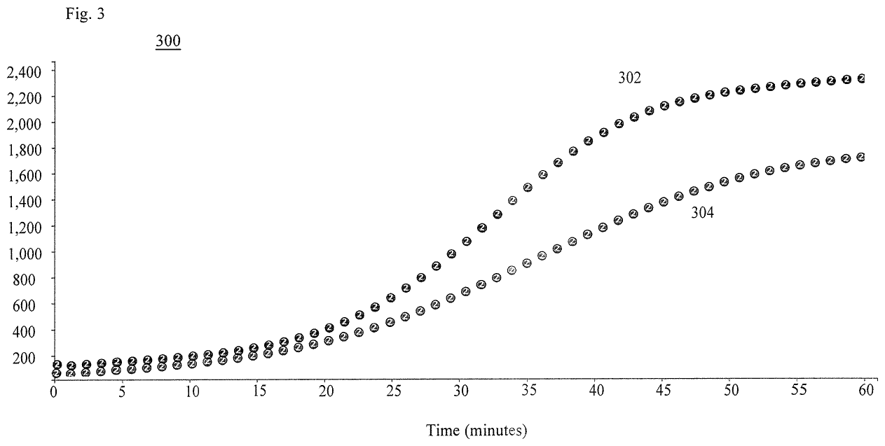

FIG. 3 demonstrates the efficacy of the method 200 deploying a treatment buffer for detecting the presence of the Sars-Cov2 RNA virus from a human naso-pharyngeal sample as a biological sample. The treatment buffer was chosen prior to collection of the biological sample that included 10% Bovine Serum Albumin, 0.4 mM EDTA of pH 8, 48 mM Guanidine isothiocyanate, and 8 mM Tris-HCl of pH 9.0. While a specific treatment buffer was used in this instance, other treatment buffers may be used.

The biological sample was collected from as a human naso-pharyngeal sample using a conventional nasal swab collected into conventional VTM solution. The biological sample collected into the conventional VTM solution was then treated with the treatment buffer, where the volume of treatment buffer was equal to the volume of biological sample collected into the viral transport media solution.

The collected biological sample having been contacted with the treatment buffer was then heated at 95 degrees Celsius for 2 minutes. Upon completion of heating, 5 microliters of the collected biological sample having been contacted with the treatment buffer and heated was then analyzed for the Sars-CoV2 RNA virus and a human internal positive control of DNA, specifically the Cox-1 (cytochrome c oxidase subunit 1 mitochondrial gene). The analysis was C-SAND with fluorescence detection, where the treatment buffer having the released biological sample was contacted with regents to perform the C-SAND analysis with fluorescence detection of the Sars-Cov2 RNA virus and Cox-1. In this example the device to carry out the analysis is the device as described in U.S. Pat. Nos. 9,568,429 and 9,810,631 titled WAVELENGTH SCANNING APPARATUS AND METHOD OF USE THEREOF was used to carry out the analysis, but other equipment capable of carrying out C-SAND analysis with fluorescence detection may be used.

The graph 300 of FIG. 3 demonstrates that the method detected both the Cox-1 positive internal control 302 and the Sars-Cov2 RNA virus 304, demonstrating that the method 200 using the treatment buffer allows detection of RNA and DNA without purification or isolation of nucleic acids from the biological sample. The units on the y-axis of the graph are fluorescence intensity.

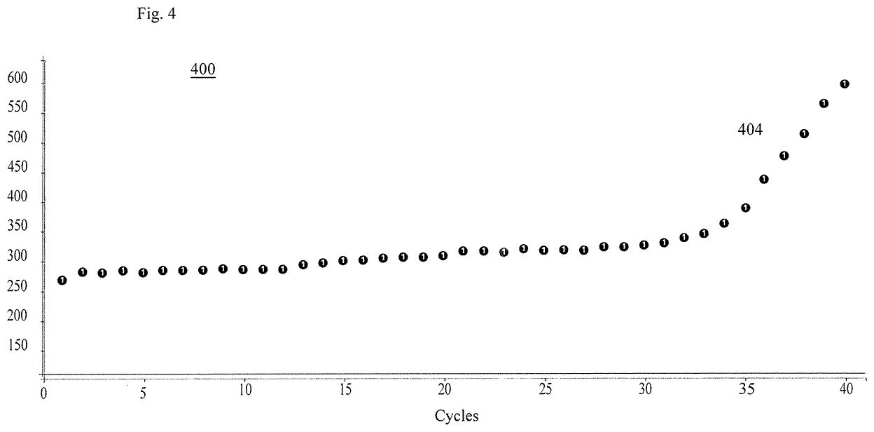

FIG. 4 demonstrates the efficacy of the method 200 using a treatment buffer for detecting the presence of the Sars-Cov2 RNA virus from a human naso-pharyngeal sample as a biological sample. The treatment buffer was chosen prior to collection of the biological sample that included 0.25 mM Sodium Citrate pH 6.7 and 2 mM TCEP-HCl. While a specific treatment buffer was used in this instance, other treatment buffers may be used.

The biological sample was collected from as a human naso-pharyngeal sample using a conventional nasal swab collected into 500 microliters of the treatment buffer. The collected biological sample contacted with the treatment buffer was then heated at 95 degrees Celsius for 2 minutes.

100 microliters of the collected biological sample contacted with the treatment buffer having been heated was then analyzed for the Sars-CoV2 RNA virus. The analysis was real-time PCR with fluorescence detection, where the collected biological sample contacted with the treatment buffer having been heated was contacted with dry regents to perform the conventional real-time PCR with fluorescence detection of the Sars-Cov2 RNA. In this example the device to carry out the analysis instance the device as described in U.S. Pat. Nos. 9,568,429 and 9,810,631 titled WAVELENGTH SCANNING APPARATUS AND METHOD OF USE THEREOF was used to carry out the analysis, but other equipment capable of real-time PCR with fluorescence detection may be used.

The graph 400 of FIG. 4 demonstrates that the method 200 detected the Sars-Cov2 RNA virus 404, demonstrating that the method 200 using the treatment buffer allows detection of RNA without purification or isolation of nucleic acids from the biological sample. The units on the y-axis of the graph are fluorescence intensity and the x-axis units indicate real-time PCR cycles.

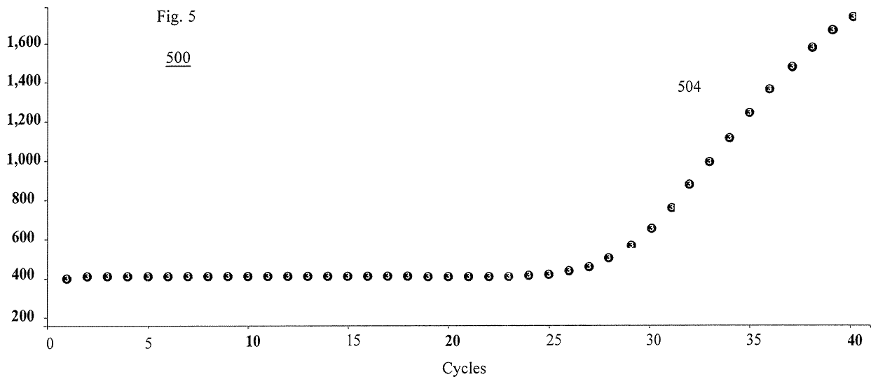

FIG. 5 demonstrates the efficacy of the method 200 deploying a treatment buffer for detecting the presence of the porcine reproductive and respiratory syndrome, also called betaarterivirus suid 1 (referred to as PRRS) RNA virus from an oral fluid sample from a pig as a biological sample. The treatment buffer was chosen prior to collection of the biological sample that included 2 mM DCTA of pH 8, 4 mM DTPA of pH 8, 1 mM EDTA of pH 8, 5 mM Sodium Citrate of pH 6.5, 2 mM TCEP-HCl. While a specific treatment buffer was used in this instance, other treatment buffers may be used.

The biological sample was collected from the chew rope from pigs as the oral fluids. The collected biological sample was then centrifuged at 2,0000× gravity in a mini centrifuge for 10 minutes to separate non-cellular debris (i.e. chew rope fragments or food particles) from the collected biological sample. The collected biological sample was treated with the treatment buffer, where the volume of treatment buffer was equal to the volume of the collected biological sample.

The collected biological sample having been contacted with the treatment buffer was then heated at 80 degrees Celsius for 10 minutes. Upon completion of heating, approximately 10 microliters of the collected biological sample having been contacted with the treatment buffer and heated was then analyzed for the PRRS virus RNA. The analysis was real-time PCR with fluorescence detection, where the collected biological sample contacted with the treatment buffer having been heated was contacted regents to perform the real-time PCR with fluorescence detection of the PRRS virus RNA. In this example the device to carry out the analysis instance the device as described in U.S. Pat. Nos. 9,568,429 and 9,810,631 titled WAVELENGTH SCANNING APPARATUS AND METHOD OF USE THEREOF was used to carry out the analysis, but other equipment capable of carrying out real-time PCR with fluorescence detection may be used.

The graph 500 of FIG. 5 demonstrates that the method detected the PRRS RNA virus 504, demonstrating that the method 200 using the treatment buffer allows detection of RNA without purification or isolation of nucleic acids from the biological sample. The units on the y-axis of the graph are fluorescence intensity, and the units of the x-axis are real-time PCR cycles.

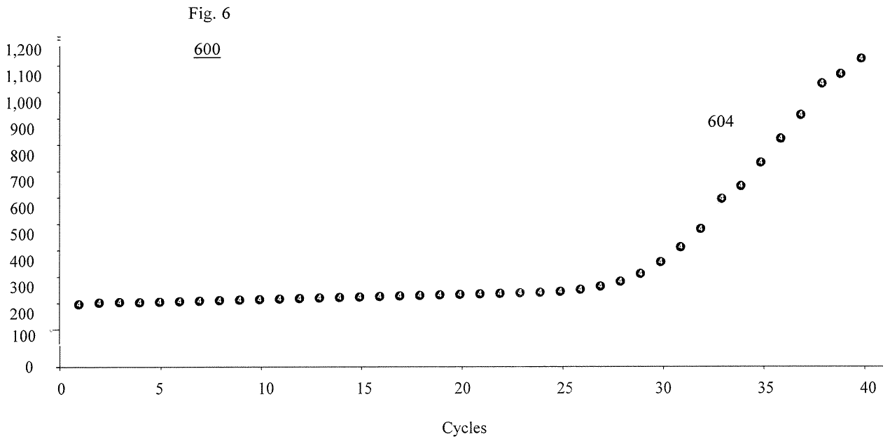

FIG. 6 demonstrates the efficacy of the method 200 deploying a treatment buffer for detecting the presence of the PRRS RNA virus from a serum sample from a pig as a biological sample. The treatment buffer was chosen prior to collection of the biological sample that included 2 mM DCTA of pH 8, 4 mM DTPA of pH 8, 1 mM EDTA of pH 8, 1 mM EGTA of pH 8, 10 mM Tris HCl of pH 9.0. While a specific treatment buffer was used in this instance, other treatment buffers may be used.

The biological sample was collected as serum and 50 microliters of the collected biological sample were treated with the treatment buffer, where the volume of treatment buffer was equal to the volume of the collected biological sample.

The collected biological sample having been contacted with the treatment buffer was then heated at 95 degrees Celsius for 2 minutes. The heated and treatment buffer contacted biological sample is then centrifuged to separate non-cellular and cellular particulate from the nucleic acids. Upon completion of centrifuging, approximately 10 microliters of the collected biological sample having been contacted with the treatment buffer and heated was then analyzed for the PRRS RNA virus. The analysis was real-time PCR with fluorescence detection, where the collected biological sample contacted with the treatment buffer having been heated was contacted regents to perform the real-time PCR with fluorescence detection of the PRRS virus RNA. In this example the device to carry out the analysis instance the device as described in U.S. Pat. Nos. 9,568,429 and 9,810,631 titled WAVELENGTH SCANNING APPARATUS AND METHOD OF USE THEREOF was used to carry out the analysis, but other equipment capable of carrying out real-time PCR analysis with fluorescence detection may be used.

The graph 600 of FIG. 6 demonstrates that the method detected the PRRS virus RNA 604, demonstrating that the method 200 using the treatment buffer allows detection of RNA without purification or isolation of nucleic acids from the biological sample. The units on the y-axis of the graph are fluorescence intensity, and the x-axis units are cycles of real-time PCR.

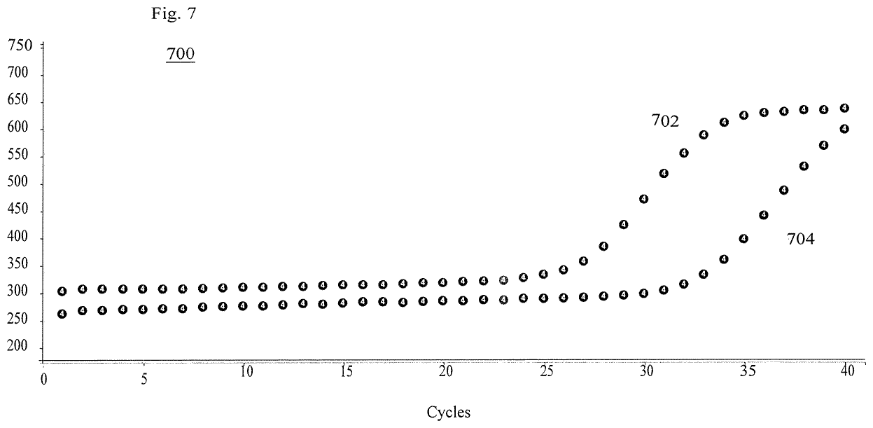

FIG. 7 demonstrates the efficacy of the method 200 deploying a treatment buffer for detecting the presence of the PRRS RNA virus from a processing fluid sample from a pig as a biological sample. The treatment buffer was chosen prior to collection of the biological sample that included 3 mM magnesium chloride, 75 mM potassium chloride, 50 mM Tris HCl of pH 9.0, and from 5.0 mM TCEP-HCl, where the buffer is adjusted to pH 8.3. While a specific treatment buffer was used in this instance, other treatment buffers may be used.

The biological sample was collected as serum and 50 microliters of the collected biological sample were treated with the treatment buffer, where the volume of treatment buffer was equal to the volume of the collected biological sample.

The collected biological sample having been contacted with the treatment buffer was then heated at 95 degrees Celsius for 2 minutes. The heated and treatment buffer contacted biological sample is then centrifuged to separate non-cellular and cellular particulate from the nucleic acids. Upon completion of centrifuging, approximately 10 microliters of the collected biological sample having been contacted with the treatment buffer and heated was then analyzed for the PRRS RNA virus and a porcine internal positive control of DNA, specifically Cox-1. The analysis was real-time PCR with fluorescence detection, where the collected biological sample contacted with the treatment buffer having been heated was contacted regents to perform the real-time PCR with fluorescence detection of the PRRS virus RNA and Cox-1. In this example the device to carry out the analysis instance the device as described in U.S. Pat. Nos. 9,568,429 and 9,810,631 titled WAVELENGTH SCANNING APPARATUS AND METHOD OF USE THEREOF was used to carry out the analysis, but other equipment capable of carrying out real-time PCR analysis with fluorescence detection may be used.

The graph 700 of FIG. 7 demonstrates that the method detected the Cox-1 positive internal control 702 and the PRRS RNA virus 704, demonstrating that the method 200 using the treatment buffer allows detection of RNA and DNA without purification or isolation of nucleic acids from the biological sample. The units on the y-axis of the graph are fluorescence intensity, and the units of the x-axis indicate cycles of real-time PCR.

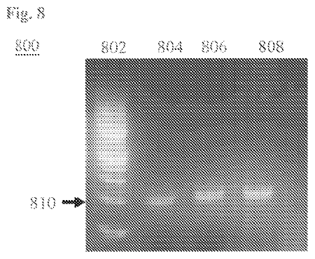

FIG. 8 demonstrates the efficacy of the method 200 deploying a treatment buffer for detecting the presence of hemochromatosis (HFE) and sickle cell anemia (HBB) genes of human DNA using oral swabs as a biological sample. The treatment buffer was chosen prior to collection of the biological sample that included 0.5 mM EDTA of pH 8, 80 mM Guanidine isothiocyanate, and 10 mM Tris-HCl of pH 9.0. While a specific treatment buffer was used in this instance, other treatment buffers may be used.

The biological sample was collected from as an oral swab using a conventional oral swab collected into conventional VTM solution. The biological sample collected into the conventional VTM solution was then treated with the treatment buffer, where the volume of treatment buffer was equal to the volume of biological sample collected into the viral transport media solution.

The collected biological sample having been contacted with the treatment buffer was then heated at 95 degrees Celsius for 2 minutes. Upon completion of heating, the collected biological sample having been contacted with the treatment buffer and heated was then analyzed for the hemochromatosis genes, in particular the HFE genes having G63D and C282Y mutations and sickle cell anemia gene, in particular the HBB gene, using a conventional PCR and gel electrophoresis protocol using a 2% agarose gel with a 100 base pair ladder.

FIG. 8 represents the results of the PCR and gel electrophoresis by a photograph of the gel 800. 802 represents the column for the 100 base-pair ladder with 810 identifying where the 200 base pair band appears on the gel. 804 is the column for identification of the PCR product specifically amplifying the C282Y mutation region, where there is a band at 216 base pairs indicating the presence of the hemochromatosis gene having the location of the C282Y mutation. 806 is the column for identification of the PCR product specifically amplifying the G63D mutation, where there is a band at 220 base pairs indicating the presence of the hemochromatosis gene having the location of the G63D mutation. 808 is the column for identification of HBB gene, where there is a band at 227 base pairs indicating the presence of the HBB gene for sickle cell anemia. FIG. 8 demonstrates that the method 200 using the treatment buffer allows detection of DNA without purification or isolation of nucleic acids from the biological sample.

1. A method of direct human, animal, microbial, and viral nucleic acid detection from a biological sample without isolation or purification of the nucleic acids prior to detection of the nucleic acids, the method comprising:
contacting a collected biological sample with a treatment buffer;
heating the collected biological sample contacted with the treatment buffer from 80 to 95 degrees Celsius from 2 to 10 minutes;
analyzing the biological sample for the nucleic acid.

2. The method of claim 1, wherein
the collected biological sample is a nasal swab from a human collected into a volume of viral transport media solution, where the nucleic acid analyzed detects the Sars-Cov2 ribonucleic acid virus.

3. The method of claim 2, wherein
the treatment buffer comprises 10% Bovine Serum Albumin, 0.4 mM ethylene diamine tetra acetic acid of pH 8, 48 mM Guanidine isothiocyanate, and 8 mM Tris(hydroxymethyl)aminomethane hydrochloride of pH 9.0 of a volume equal to the volume of collected biological sample.

4. The method of claim 3, wherein
the heating comprises heating the collected biological sample contacted with the treatment buffer at 95 degrees Celsius for 2 minutes.

5. The method of claim 1, wherein
the collected biological sample is an oral swab from a human collected into 500 microliters of the treatment buffer, where the nucleic acid analyzed detects the Sars-Cov2 ribonucleic acid virus.

6. The method of claim 5, wherein
the treatment buffer comprises 0.25 mM Sodium Citrate pH 6.7 and 2 mM Tris(2-carboxyethyl)phosphine hydrochloride, and wherein the heating comprises heating the collected biological sample contacted with the treatment buffer at 95 degrees Celsius for 2 minutes.

7. The method of claim 1, wherein
the collected biological sample is an oral fluid sample of a porcine chew rope, where the nucleic acid analyzed detects the porcine reproductive and respiratory syndrome ribonucleic acid virus, wherein
the collected biological sample is centrifuged at 2,000× gravity in a mini centrifuge for 10 minutes.

8. The method of claim 7, wherein
the treatment buffer comprises a volume equal to a volume of the collected biological sample of 2 mM 1,2-Cyclohexanedinitrilotetraacetic acid of pH 8, from 4 mM diethylenetriaminepentaacetic acid of pH 8, 1 mM ethylene diamine tetra acetic acid of pH 8, 5 mM Sodium Citrate of pH 6.5, 2 mM Tris(2-carboxyethyl)phosphine hydrochloride.

9. The method of claim 8, wherein
the heating comprises heating the collected biological sample contacted with the treatment buffer at 80 degrees Celsius for 10 minutes.

10. The method of claim 1, wherein
the collected biological sample is 50 microliters of porcine serum where the nucleic acid analyzed detects the porcine reproductive and respiratory syndrome ribonucleic acid virus.

11. The method of claim 10, wherein
the treatment buffer is 50 microliters of 2 mM 1,2-Cyclohexanedinitrilotetraacetic acid of pH 8, 4 mM diethylenetriaminepentaacetic acid of pH 8, 1 mM ethylene diamine tetra acetic acid of pH 8, 1 mM ethylene glycol-bis(β-aminoethyl ether)-N,N,N′,N′-tetraacetic acid of pH 8, 10 mM Tris(hydroxymethyl)aminomethane hydrochloride of pH 9.0, and wherein
the heating comprises heating the collected biological sample contacted with the treatment buffer at 95 degrees Celsius for 2 minutes.

12. The method of claim 1, wherein
the collected biological sample is 50 microliters of porcine processing fluid where the nucleic acid analyzed detects the porcine reproductive and respiratory syndrome ribonucleic acid virus.

13. The method of claim 12, wherein
the treatment buffer comprises 50 microliters of 3 mM magnesium chloride, 75 mM potassium chloride, 50 mM Tris(hydroxymethyl)aminomethane hydrochloride of pH 9.0, and from 5.0 mM mM Tris(2-carboxyethyl)phosphine hydrochloride, where the treatment buffer is adjusted to pH 8.3.

14. The method of claim 13, wherein
the heating comprises heating the collected biological sample contacted with the treatment buffer at 95 degrees Celsius for 2 minutes.

15. The method of claim 1, wherein
the collected biological sample is an oral swab from a human collected into a volume of universal transport media, where the nucleic acid analyzed is a hemochromatosis (HFE) gene having a G63D mutation.

16. The method of claim 12, wherein
the treatment buffer is a volume equal to the volume of universal transport media comprising 0.5 mM ethylene diamine tetra acetic acid of pH 8, 80 mM Guanidine isothiocyanate, and 10 mM Tris(hydroxymethyl)aminomethane hydrochloride of pH 9.0, and wherein
the heating comprises heating the collect biological sample contacted with the treatment buffer at 95 degrees Celsius for 2 minutes.

17. A method of direct human, animal, microbial, and viral nucleic acid detection from a biological sample without isolation or purification of nucleic acids prior to detection of the nucleic acids, the method comprising:
contacting a collected biological sample with a treatment buffer, the treatment buffer comprising at least one chelating agent and at least one buffering agent;
heating the collected biological sample contacted with the treatment buffer from 80 to 95 degrees Celsius from 2 to 10 minutes;
analyzing the biological sample for the nucleic acid.

18. The method of claim 17, wherein
the chelating agent is selected from the group consisting of from 0.4 to 1 milliMolar (mM) ethylene diamine tetra acetic acid of pH 8.0, 2 mM 1,2-Cyclohexanedinitrilotetraacetic acid of pH 8, from 2-4 mM diethylenetriaminepentaacetic acid of pH 8, from 0.25 to 5 mM Tris(2-carboxyethyl)phosphine hydrochloride, and 1 mM ethylene glycol-bis(β-aminoethyl ether)-N,N,N′,N′-tetraacetic acid, and combinations thereof.

19. The method of claim 18, wherein
the buffering agent is selected from the group consisting of from 8 to 50 mM Tris(hydroxymethyl)aminomethane hydrochloride, 3 mM magnesium chloride, and 75 mM potassium chloride, 5 mM sodium citrate of pH from 6.5 to 6.7, and combinations thereof.

20. The method of claim 19, wherein
the treatment buffer further comprises a lysis agent selected from the group consisting of from 48 to 250 mM guanidine isothicynate, 2 mM Tris(2-carboxyethyl)phosphine hydrochloride, and combinations thereof.
